(19) United States
(12) Patent Application Publication
(10) Pub. No.: US 2026/0294360 A1
(43) Pub. Date: Oct. 1, 2026

(54) CLADDING PART FOR A GANTRY OF A COMPUTED TOMOGRAPHY DEVICE

(71) Applicant: Siemens Healthineers AG, Forchheim (DE)

(72) Inventors: Christoph EICHNER, Erlangen (DE); Jens FEHRE, Hausen (DE)

(73) Assignee: Siemens Healthineers AG, Forchheim (DE)

(21) Appl. No.: 19/632,816

(22) Filed: Mar. 30, 2026

(30) Foreign Application Priority Data
Mar. 31, 2025 (DE) 20 2025 101 738.5

(51) Int. Cl. A61B 6/03; A61B 6/00; A61B 6/10

(52) U.S. Cl. CPC A61B 6/035; A61B 6/107; A61B 6/4447

(57) ABSTRACT

A housing part has an annular disk molded part and a cylindrical jacket molded part. The annular disk molded part and the cylindrical jacket molded part are arranged coaxially relative to a system axis such that an inner circumference of the annular disk molded part is attached to a base of the cylindrical jacket molded part. The annular disk molded part has a first wall region and a second wall region, which lie opposite one another relative to the system axis, abut the inner circumference of the annular disk molded part and extend substantially perpendicular to the system axis. The annular disk molded part has a reinforcement structure extending substantially parallel to the system axis away from the cylindrical jacket molded part, such that the first wall region and the second wall region are between the reinforcement structure and the cylindrical jacket molded part relative to the system axis.

### CROSS-REFERENCE TO RELATED APPLICATION(S)

The present application claims priority under 35 U.S.C. § 119 to German Patent Application No. 20 2025 101 738.5, filed Mar. 31, 2025, the entire contents of which is incorporated herein by reference.

### FIELD

One or more example embodiments of the present invention relate to a housing part (also referred to as a cladding part or cover part) for a gantry of a computed tomography device. One or more example embodiments of the present invention further relate to a gantry and to a computed tomography device.

### BACKGROUND

Housing parts of a gantry of a computed tomography device should, on the one hand, have a low weight and a low installation space requirement, while on the other hand being as stable as possible, in particular in order to comply with specified safety distances from rotating components of the gantry even under load.

In this context, DE 103 34 326 A1, DE 10 2007 010 061 A1, DE 10 2016 208 328 A1, DE 20 2021 103 613 U1 and DE 20 2024 104 403 U1 are cited as prior art.

### SUMMARY

An object of one or more example embodiments of the present invention is to provide an alternative to conventional gantry housing parts in computed tomography devices, in particular with regard to the aforementioned requirements. The independent claims relate to solutions for this object. The dependent claims relate to particular embodiments of these solutions. Independent of the grammatical term usage, individuals with male, female or other gender identities are included within the term.

One or more example embodiments of the present invention relate to a housing part (also referred to as cladding part or cover part) for a gantry of a computed tomography device,
wherein the housing part has an annular disk molded part and a cylindrical jacket molded part,
wherein the annular disk molded part and the cylindrical jacket molded part are arranged coaxially in relation to a system axis in such a manner that an inner circumference of the annular disk molded part is attached to a base of the cylindrical jacket molded part,
wherein the annular disk molded part has a first wall region and a second wall region, which lie opposite one another in relation to the system axis, abut the inner circumference of the annular disk molded part and extend substantially perpendicular to the system axis in a planar manner,
wherein the annular disk molded part has a reinforcement structure, which extends substantially in parallel with the system axis in a planar manner away from the cylindrical jacket molded part, in such a manner that the first wall region and the second wall region are located between the reinforcement structure and the cylindrical jacket molded part in relation to the system axis.

Furthermore, a housing part for a gantry of a computed tomography device is hereby disclosed,
wherein the housing part has an annular disk molded part and a cylindrical jacket molded part,
wherein the annular disk molded part and the cylindrical jacket molded part are arranged coaxially in relation to a system axis in such a manner that an inner circumference of the annular disk molded part is attached to a base of the cylindrical jacket molded part,
wherein the annular disk molded part has a first wall region and a second wall region, which lie opposite one another in relation to the system axis, abut the inner circumference of the annular disk molded part and extend substantially perpendicular to the system axis in a planar manner,
wherein the annular disk molded part has a reinforcement structure.

In particular, it can be provided that the reinforcement structure extends substantially in parallel with the system axis in a planar manner away from the cylindrical jacket molded part and/or that the first wall region and the second wall region are located between the reinforcement structure and the cylindrical jacket molded part in relation to the system axis.

In particular, it can be provided that the reinforcement structure extends substantially in parallel with the system axis in a planar manner away from the cylindrical jacket molded part, in such a manner that the first wall region and the second wall region are located between the reinforcement structure and the cylindrical jacket molded part in relation to the system axis.

The inner circumference of the annular disk molded part and the base of the cylindrical jacket molded part can be adhered to one another, for example. The wall thickness of the annular disk molded part and/or the cylindrical jacket molded part can be less than 30 mm, in particular less than 20 mm, for example. In particular, it can be provided that the first wall region, the second wall region and the reinforcement structure transition into one another in one piece.

The reinforcement structure causes an increased rigidity of the housing part. This makes it possible to reduce a safety distance between the annular disk molded part and a trajectory of rotating components of the gantry. This makes it possible to bring a patient closer to the scan plane, and thus increase the scan depth, in a head computed tomography device. Due to a high scan depth, it is possible for more of the head and neck region of the patient to be scanned, whereby the possible examination region is increased.

The scan depth can be increased by bringing the housing part closer to the beam geometry. In doing so, it is possible to comply with a safety distance between the rotating part of the gantry, in which the beam geometry is located, and the housing part, so that the housing part cannot be pushed into the rotating parts of the gantry due to loads that occur during the clinical workflow.

Due to the increased rigidity, the amount of material and thus the total weight of the housing part can be reduced, for example, to the point that a manual removal of the housing part for maintenance purposes can be performed by a person.

One embodiment provides that the reinforcement structure has a first partial structure, wherein the first partial structure extends in a planar manner in a first reinforcement plane, which is substantially in parallel with the system axis. In particular, it can be provided that the first partial structure abuts the inner circumference of the annular disk molded part and/or that the first reinforcement plane runs substantially tangentially to a peripheral surface of the cylindrical jacket molded part.

One embodiment provides that the reinforcement structure has a second partial structure, wherein the second partial structure extends in a planar manner in a second reinforcement plane, which is in parallel with the first reinforcement plane, wherein the first wall region and the second wall region are located between the first reinforcement plane and the second reinforcement plane in relation to a direction that is perpendicular to the system axis.

One embodiment provides that the annular disk molded part and/or the cylindrical jacket molded part is/are produced from a fiber-reinforced composite material, in particular a carbon fiber-reinforced composite material.

The molded parts can be produced by an autoclave method and/or a deep drawing method, for example. Housing parts with a high rigidity can be produced from fiber-reinforced composite materials, so that the safety distance from the trajectory of the rotating components can be reduced and thus the housing part can be moved closer to the beam geometry. This makes it possible to increase the scan depth. Furthermore, through the use of fiber-reinforced composite materials, the wall thickness of the housing part can be reduced significantly. As a result, the cover can be brought even closer to the beam geometry, which causes a further increase in the scan depth.

One or more example embodiments of the present invention further relate to a gantry for a computed tomography device, wherein the gantry has a cladding for delineating an inner region of the gantry from a surrounding area of the gantry, wherein the cladding has the housing part according to one or more example embodiments of the present invention, wherein the gantry has an opening, wherein an examination object can be introduced into the opening along the system axis, wherein the cylindrical jacket molded part delineates the inner region of the gantry from the opening.

One embodiment provides that a radiation protection material fits to a side of the annular disk molded part, which faces toward the inner region of the gantry, in a planar manner. The radiation protection material can be embodied in the form of a film, in particular a lead film, for example, and/or can follow a step-shaped course of the housing part. The surrounding area of the gantry, in which an operator or a patient can be located for example, can be protected from scattered radiation by the radiation protection material. The relatively high weight of the radiation protection material can be partially compensated for by a relatively light weight of the housing part.

One embodiment provides that the gantry has a projection data acquisition system, wherein the projection data acquisition system is configured for capturing projection data of the examination object introduced into the opening, wherein a distance from the reinforcement structure to the system axis, in particular a distance from the first reinforcement plane and/or the second reinforcement plane to the system axis, is smaller than a distance from the projection data acquisition system to the system axis.

In particular, the examination object can be a head of a patient. The shoulders of the patient can be wider than the opening, for example. In particular, a tight fit between the shoulders of the patient and the first wall region and the second wall region can counteract a further introduction of the head of the patient into the opening along the system axis with a tight fit.

The distance of the projection data acquisition system from the system axis can be, for example, a distance of an X-ray source of the projection data acquisition system and/or a distance of an X-ray detector of the projection data acquisition system from the system axis.

One embodiment provides that the reinforcement structure has a window, such that an optical axis of an optical component of the computed tomography system can penetrate the window if the optical component is located in the inner region of the gantry.

The optical component can be, for example, a camera for observing the examination object and/or the patient and/or a laser unit for the laser-based positioning of the examination object. In particular, it can be provided that the first wall region and the second wall region are located between the optical component and the cylindrical jacket molded part in relation to the system axis. In particular, it can be provided that the optical axis is substantially perpendicular to the system axis and/or that the first wall region and the second wall region are located between the optical axis and the cylindrical jacket molded part in relation to the system axis.

One embodiment provides that the housing part has a hat-like bulge toward the surrounding area of the gantry, wherein the hat-like bulge widens toward the reinforcement structure, in particular toward the first partial structure of the reinforcement structure, wherein a partial region of the inner region of the gantry is delineated from the surrounding area of the gantry by the hat-like bulge and the reinforcement structure, wherein a retainer for fastening the optical component to the reinforcement structure is located in the partial region of the inner region of the gantry.

One or more example embodiments of the present invention further relate to a computed tomography device, in particular a mobile head computed tomography device, having a gantry according to one or more example embodiments of the present invention.

One embodiment provides that the gantry has a first gantry part, a second gantry part and a third gantry part, wherein the first gantry part has a rotatably mounted rotor with a projection data acquisition system, wherein the third gantry part has at least one section of the opening, wherein the retaining structure is connected to the third gantry part via the pivot apparatus and is mounted such that it can pivot relative to the third gantry part about the pivot axis. The projection data acquisition system can have, for example, an X-ray source and an X-ray detector, which interacts with the X-ray source.

In this context, the first gantry part can be mounted such that it can move relative to the second gantry part and relative to the third gantry part in such a manner that a translation movement of the first gantry part relative to the second gantry part and relative to the third gantry part can be carried out, while at the same time the second gantry part and the third gantry part are lying still relative to the examination object and the radiation protection apparatus is lying still relative to the examination object and relative to the at least one section of the opening when the examination object is located in the opening.

The examination object can be, for example, a body part of a patient, in particular a head of a patient. The computed tomography device can be embodied, in particular, as a head computed tomography device and/or as a mobile computed tomography device. The patient can, for example, involve a person, in particular a baby, or an animal. A longitudinal axis of the patient can, for example, be parallel to the system axis, in particular identical to it, or skewed in relation thereto, or can intersect the system axis. The examination object can, for example, be an object, in particular a phantom for the calibration of the computed tomography device.

In the context of the present invention, features which are described in relation to different embodiments of the present invention and/or different claim categories (method, use, apparatus, system, arrangement, etc.) can be combined to form further embodiments of the present invention. For example, a claim which relates to an apparatus can also be developed with features which are described or claimed in relation to a method, and vice versa. Functional features of a method can be carried out by way of correspondingly embodied physical components. The use of the indefinite article “a” or “an” does not preclude that the relevant feature can also be present plurally.

### BRIEF DESCRIPTION OF THE DRAWINGS

Features of the present invention will be explained in the following on the basis of examples, with reference to the accompanying figures. The representation in the figures is schematic, greatly simplified and not necessarily to scale.

FIGS. 1 to 4 show a housing part for a gantry of a computed tomography device in various views.

FIGS. 5 and 6 show a computed tomography device with a gantry.

### DETAILED DESCRIPTION

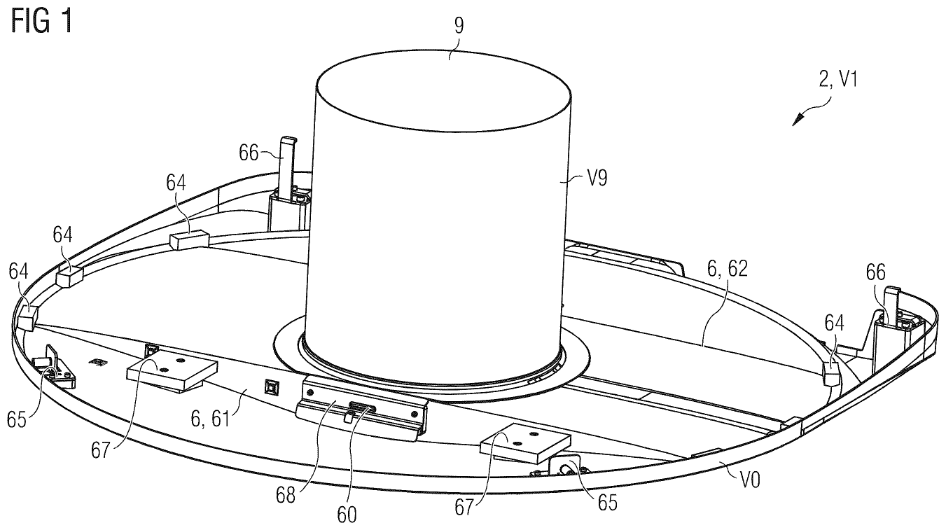

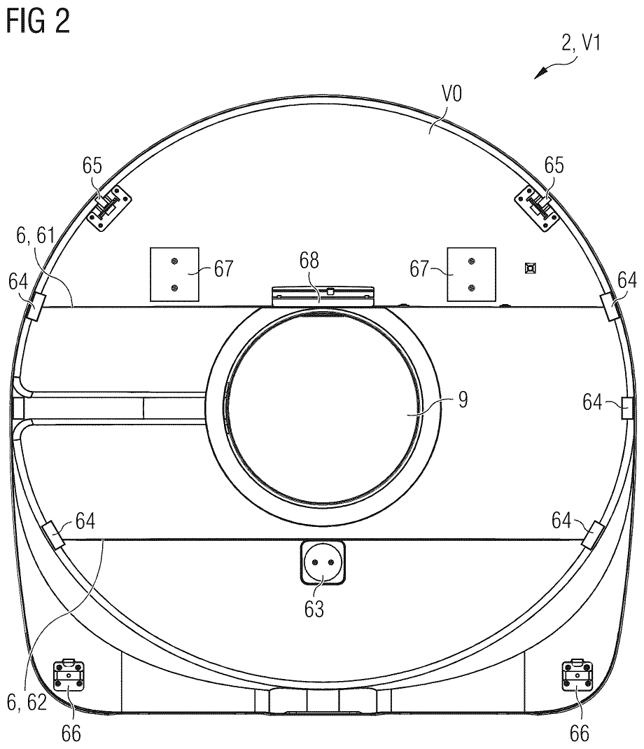

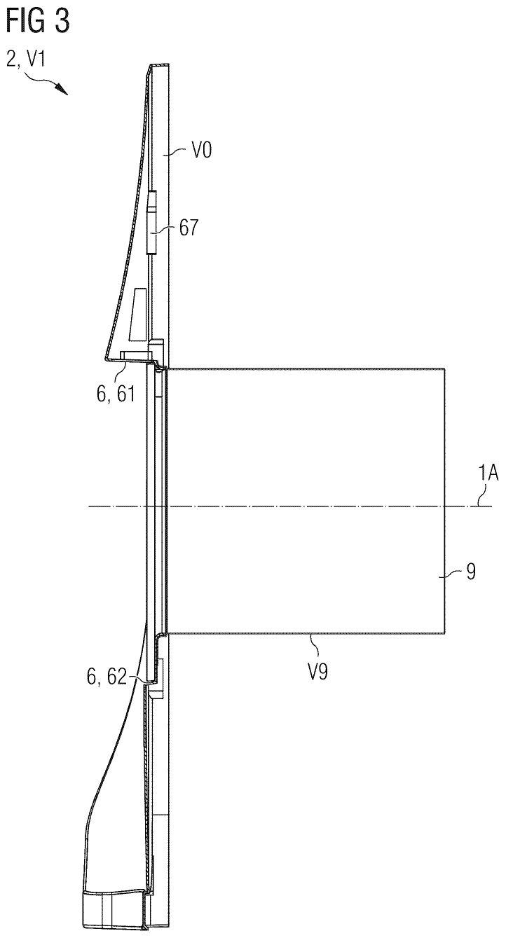

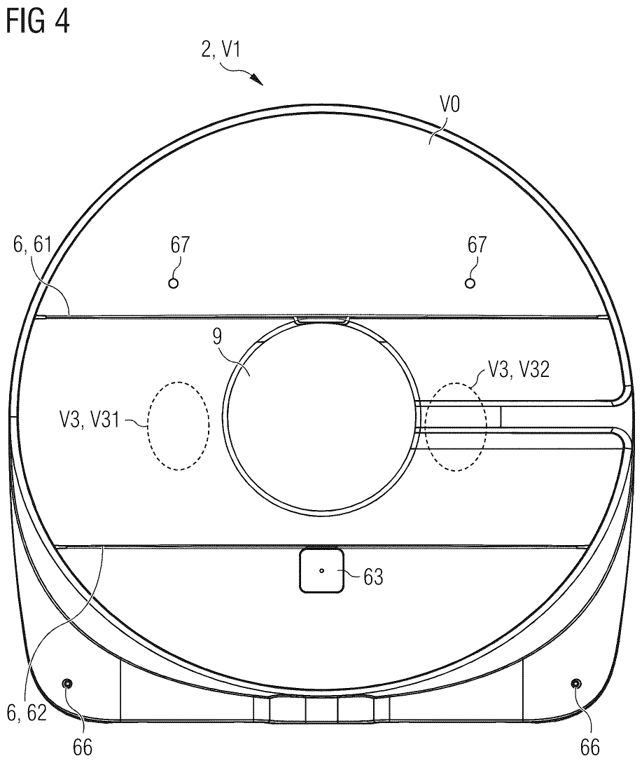

FIGS. 1 to 4 show the housing part V1 for a gantry 20 of a computed tomography device 1 in various views,
wherein the housing part V1 has an annular disk molded part V0 and a cylindrical jacket molded part V9,
wherein the annular disk molded part V0 and the cylindrical jacket molded part V9 are arranged coaxially in relation to a system axis 1A in such a manner that an inner circumference of the annular disk molded part V0 is attached to a base of the cylindrical jacket molded part V9,
wherein the annular disk molded part V0 has a first wall region V31 and a second wall region V32, which lie opposite one another in relation to the system axis 1A, abut the inner circumference of the annular disk molded part V0 and extend substantially perpendicular to the system axis 1A in a planar manner,
wherein the annular disk molded part V0 has a reinforcement structure 6, which extends substantially in parallel with the system axis 1A in a planar manner away from the cylindrical jacket molded part V9, in such a manner that the first wall region V31 and the second wall region V32 are located between the reinforcement structure 6 and the cylindrical jacket molded part V9 in relation to the system axis 1A.

Blocks 64, to which a further part V2 of the cladding V can be attached with a positive fit, are fastened, in particular adhered, to the outer circumference of the annular disk molded part V0. Via the blocks 64, the mechanical stresses that result from the connection of the housing part V1 to the further part V2 of the cladding V, can be distributed evenly over the annular disk molded part. Via the fastening mechanism, device and/or means 65, 66 (also referred to as a fastener), the housing part V1 can be fastened to the further part V2 of the cladding. Additionally, a retainer 67 for a retaining structure 7 of a radiation protection apparatus 75 is fastened to the housing part V1. Additionally fastened to the housing part V1 is a connecting element 63 for connection, in particular detachable electromagnetic connection, of the housing part V1 to a retaining column 32, on which the upper body positioning plate 15 and the head tray 19 are also arranged. Together with the constituent parts 63, 64, 65, 66, 67 and 68, the housing part V1 forms an integrated cladding and retaining structure 2 of the gantry 20.

The example shown provides that the reinforcement structure 6 has a first partial structure 61, wherein the first partial structure 61 extends in a planar manner in a first reinforcement plane, which is substantially in parallel with the system axis 1A.

The example shown provides that the reinforcement structure 6 has a second partial structure 62, wherein the second partial structure 62 extends in a planar manner in a second reinforcement plane, which is in parallel with the first reinforcement plane, wherein the first wall region V31 and the second wall region V32 are located between the first reinforcement plane and the second reinforcement plane in relation to a direction y that is perpendicular to the system axis 1A.

The example shown provides that the annular disk molded part V0 and/or the cylindrical jacket molded part V9 is/are produced from a fiber-reinforced composite material, in particular a carbon fiber-reinforced composite material.

The dividing wall V3 has the first wall region V31 and the second wall region V32. Due to a tight fit between the shoulders of the patient and the first wall region V31 and the second wall region V32, stress can be caused on the dividing wall V3 when the head of the patient is introduced into the opening. Similar stress can occur if an operator leans on the dividing wall V3. The reinforcement structure 6 counteracts a bending of the dividing wall V3 under such stresses, meaning that the dividing wall V3 can be embodied with thin walls and/or a small safety distance in relation the rotating components can be provided, in order to increase the scan depth.

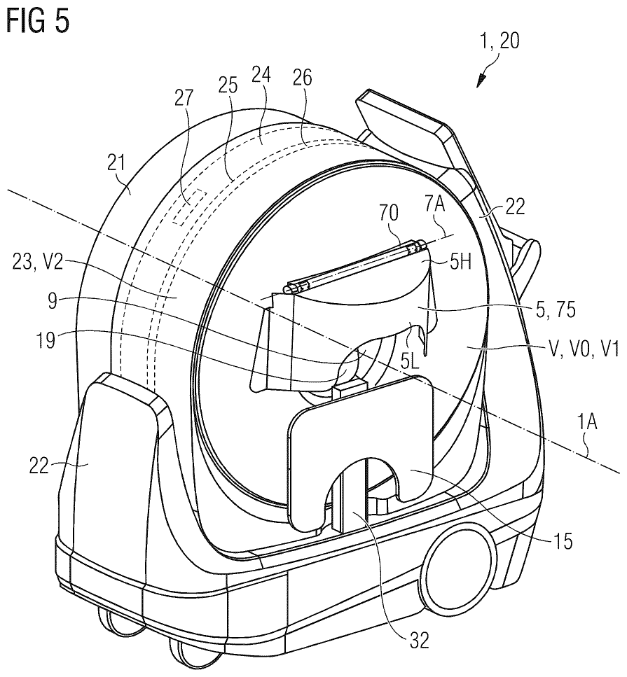

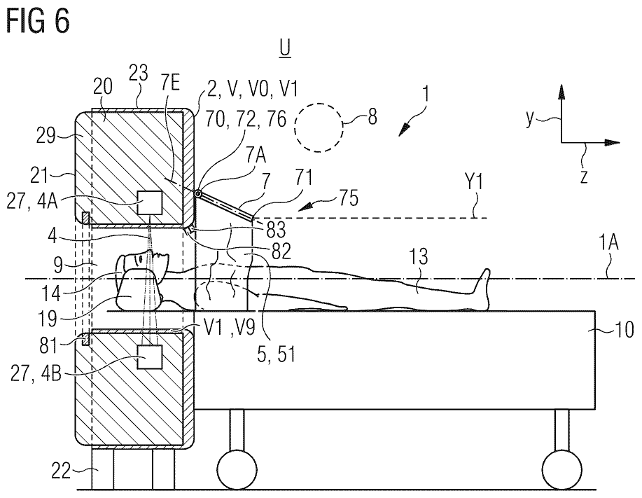

FIGS. 5 and 6 show the computed tomography device 1 with the gantry 20 and a radiation protection apparatus 75 for covering the opening 9. The example shown provides that the gantry 20 has a cladding V for delineating an inner region 29 of the gantry 20 from a surrounding area U of the gantry 20, wherein the cladding V has the housing part V1 according to the example shown, wherein the gantry 20 has an opening 9, wherein an examination object 14 can be introduced into the opening 9 along the system axis 1A, wherein the cylindrical jacket molded part V9 delineates the inner region 29 of the gantry 20 from the opening 9.

The example shown provides that a radiation protection material fits to a side of the annular disk molded part V0, which faces toward the inner region 29 of the gantry 20, in a planar manner.

The example shown provides that the gantry 20 has a projection data acquisition system 27, wherein the projection data acquisition system 27 is configured for capturing projection data of the examination object 14 introduced into the opening 9, wherein a distance from the reinforcement structure 6 to the system axis 1A, in particular a distance from the first reinforcement plane and/or the second reinforcement plane to the system axis 1A, is smaller than a distance from the projection data acquisition system 27 to the system axis 1A.

The example shown provides that the reinforcement structure 6 has a window 60, such that an optical axis of an optical component of the computed tomography system 1 can penetrate the window 60 if the optical component is located in the inner region of the gantry 20.

The example shown provides that the housing part V1 has a hat-like bulge toward the surrounding area U of the gantry 20, wherein the hat-like bulge widens toward the reinforcement structure 6, in particular toward the first partial structure 61 of the reinforcement structure 6, wherein a partial region of the inner region of the gantry 20 is delineated from the surrounding area U of the gantry 20 by the hat-like bulge and the reinforcement structure 6, wherein a retainer 68 for fastening the optical component to the reinforcement structure 6 is located in the partial region of the inner region of the gantry 20.

The examination object 14 is the head of the patient 13. The computed tomography device 1 is a mobile head computed tomography device. The computed tomography device 1 further has a head tray 19, in which the head of the patient 13 can be accommodated, and an upper body positioning plate 15, on which the upper body of the patient 13 can be positioned.

The gantry 20 has a first gantry part 21, a second gantry part 22 and a third gantry part 23, wherein the first gantry part 21 has a rotatably mounted rotor 24 with a projection data acquisition system 27, wherein the third gantry part 23 has at least one section of the opening 9. The retaining structure 7 is connected to the third gantry part 23 via the pivot apparatus 70 and is mounted such that it can pivot relative to the third gantry part 23 about the pivot axis 7A. The third gantry part 23 has the housing part V1.

The first gantry part 21 is mounted such that it can move relative to the second gantry part 22 and relative to the third gantry part 23 in such a manner that a translation movement of the first gantry part 21 relative to the second gantry part 22 and relative to the third gantry part 23 can be carried out, while at the same time the second gantry part 22 and the third gantry part 23 are lying still relative to the examination object 14 and the radiation protection apparatus 75 is lying still relative to the examination object 14 and relative to the at least one section of the opening 9 when the examination object 14 is located in the opening 9.

The first gantry part 21 has a pivot bearing 25 and a supporting structure 26, wherein the rotor 24 is connected to the supporting structure 26 via the pivot bearing 25 and is mounted such that it can rotate relative to the supporting structure 26 about the system axis 1A. The projection data acquisition system 27 has the X-ray source 4A for generating the X-ray radiation 4 and the X-ray detector 4B for detecting the X-ray radiation 4. The cylindrical jacket molded part V9 is located in the beam path of the X-ray radiation 4.

FIG. 6 shows the computed tomography device 1 with the gantry 20. The computed tomography device 1 has the patient bed 10 for positioning the patient 13. The gantry 20 has an inner region 29 and a cladding V for delineating the inner region 29 from a surrounding area U. The example shown provides that the upper region 5H of the radiation protection curtain 5 rests against the retaining structure 7 in such a manner that the upper region 5H of the radiation protection curtain 5 shields a region 8 of a surrounding area U of the gantry 20 from scattered radiation of an X-ray radiation 4 that is directed toward the region 8 of the surrounding area U of the gantry 20, coming from the opening 9, and penetrates the retaining structure 7, in particular if the retaining structure 7 is located in the shown position of the retaining structure 7.

The retaining structure 7 extends in a planar manner in a cover plane 7E, wherein the cover plane 7E is substantially parallel to the pivot axis 7A. The system axis 1A is horizontal and parallel to the horizontal direction z. The pivot axis 7A is horizontal and perpendicular to the horizontal direction z. The pivot axis 7A is located above the opening 9. The radiation protection curtain 5 is arranged on the retaining structure 7, in particular is fastened to the retaining structure 7, wherein a lower region 5L of the radiation protection curtain 5 hangs down from the retaining structure 7, wherein an upper region 5H of the radiation protection curtain 5 rests against the retaining structure 7. The pivot apparatus 70 has the friction hinge 76. A first side part 51 of the radiation protection curtain 5 extends from the gantry 20 up to a first edge 71 of the retaining structure 7. Y1 is the vertical position of the first edge 71 of the retaining structure 7. In relation to the vertical direction y, an intermediate space is located between the patient 13 and the retaining structure 7. As a result, the risk of the patient 13 colliding with the retaining structure 7 is reduced. The computed tomography device 1 further has an illumination system 81, which is configured to illuminate the opening 9.

The computed tomography device 1 further has a camera 82, which is configured to optically capture the examination object 14 at least when the examination object 14 is located in the opening 9 and/or immediately in front of the opening 9. The computed tomography device 1 further has a laser unit 83 for the laser-based positioning of the examination object 14.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

What is claimed is:

1. A housing part for a gantry of a computed tomography device, the housing part comprising:
an annular disk molded part; and
a cylindrical jacket molded part;
wherein the annular disk molded part and the cylindrical jacket molded part are arranged coaxially relative to a system axis such that an inner circumference of the annular disk molded part is attached to a base of the cylindrical jacket molded part;
wherein the annular disk molded part has a first wall region and a second wall region, which lie opposite one another relative to the system axis, abut the inner circumference of the annular disk molded part, and extend substantially perpendicular to the system axis in a planar manner; and
wherein the annular disk molded part has a reinforcement structure, which extends substantially parallel to the system axis in a planar manner away from the cylindrical jacket molded part, such that the first wall region and the second wall region are located between the reinforcement structure and the cylindrical jacket molded part relative to the system axis.

2. The housing part as claimed in claim 1, wherein
the reinforcement structure has a first partial structure, and
the first partial structure extends in a planar manner in a first reinforcement plane, which is substantially parallel to the system axis.

3. The housing part as claimed in claim 2, wherein
the reinforcement structure has a second partial structure,
the second partial structure extends in a planar manner in a second reinforcement plane, which is parallel to the first reinforcement plane, and
the first wall region and the second wall region are located between the first reinforcement plane and the second reinforcement plane relative to a direction that is perpendicular to the system axis.

4. The housing part as claimed in claim 1, wherein at least one of the annular disk molded part or the cylindrical jacket molded part is/are produced from a fiber-reinforced composite material.

5. A gantry for a computed tomography device, the gantry comprising:
a cladding to delineate an inner region of the gantry from a surrounding area of the gantry, wherein
the cladding includes the housing part as claimed in claim 1,
the gantry has an opening configured to receive an examination object introduced into the opening along the system axis, and
the cylindrical jacket molded part delineates the inner region of the gantry from the opening.

6. The gantry as claimed in claim 5, wherein a radiation protection material is configured to fit to a side of the annular disk molded part, which faces the inner region of the gantry, in a planar manner.

7. The gantry as claimed in claim 5, wherein
the gantry has a projection data acquisition system,
the projection data acquisition system is configured to capture projection data of the examination object introduced into the opening, and
a distance from the reinforcement structure to the system axis is smaller than a distance from the projection data acquisition system to the system axis.

8. The gantry as claimed in claim 5, wherein the reinforcement structure has a window, such that an optical axis of an optical component of the computed tomography device penetrates the window when the optical component is located in the inner region of the gantry.

9. The gantry as claimed in claim 8, wherein
the housing part has a hat-like bulge toward the surrounding area of the gantry,
the hat-like bulge widens toward the reinforcement structure,
a partial region of the inner region of the gantry is delineated from the surrounding area of the gantry by the hat-like bulge and the reinforcement structure, and
a retainer to fasten the optical component to the reinforcement structure is located in the partial region of the inner region of the gantry.

10. A computed tomography device comprising:
the gantry as claimed in claim 5.

11. The housing part as claimed in claim 4, wherein the fiber-reinforced composite material is a carbon fiber-reinforced composite material.

12. The gantry as claimed in claim 7, wherein the distance from the reinforcement structure to the system axis is a distance from at least one of a first reinforcement plane or a second reinforcement plane to the system axis.

13. The gantry as claimed in claim 9, wherein the hat-like bulge widens toward a first partial structure of the reinforcement structure.

14. The computed tomography device as claimed in claim 10, wherein the computed tomography device is a mobile head computed tomography device.

15. The housing part as claimed in claim 2, wherein at least one of the annular disk molded part or the cylindrical jacket molded part is/are produced from a fiber-reinforced composite material.

16. The housing part as claimed in claim 3, wherein at least one of the annular disk molded part or the cylindrical jacket molded part is/are produced from a fiber-reinforced composite material.

17. The gantry as claimed in claim 6, wherein
the gantry has a projection data acquisition system,
the projection data acquisition system is configured to capture projection data of the examination object introduced into the opening, and
a distance from the reinforcement structure to the system axis is smaller than a distance from the projection data acquisition system to the system axis.

18. The gantry as claimed in claim 6, wherein the reinforcement structure has a window, such that an optical axis of an optical component of the computed tomography device penetrates the window when the optical component is located in the inner region of the gantry.

19. The gantry as claimed in claim 7, wherein the reinforcement structure has a window, such that an optical axis of an optical component of the computed tomography device penetrates the window when the optical component is located in the inner region of the gantry.

20. A gantry for a computed tomography device, the gantry comprising:
a cladding to delineate an inner region of the gantry from a surrounding area of the gantry, wherein
the cladding includes the housing part as claimed in claim 3,
the gantry has an opening configured to receive an examination object introduced into the opening along the system axis, and
the cylindrical jacket molded part delineates the inner region of the gantry from the opening.
